(19) United States
(12) Patent Application Publication
(10) Pub. No.: US 2018/0155671 A1
(43) Pub. Date: Jun. 7, 2018

(54) AIRCRAFT FUEL TANK WITH A VISUAL CONTROL SYSTEM INSIDE THE TANK, RELATED AIRCRAFT AND VISUAL CONTROL METHOD

(71) Applicant: DASSAULT AVIATION, Paris (FR)

(72) Inventors: Jean-Philippe DUPONT, Merignac (FR); Marc CATHELAIN, Martignas-Sur-Jalle (FR)

(21) Appl. No.: 15/804,227

(22) Filed: Nov. 6, 2017

(30) Foreign Application Priority Data
Nov. 9, 2016 (FR) FR 16 01596

(51) Int. Cl. C12M 1/34; B64D 37/02; C12Q 1/04; G01F 23/292

(52) U.S. Cl. CPC C12M 41/36; G01F 23/2922; C12Q 1/04; B64D 37/02

(57) ABSTRACT

Aircraft fuel tank, which has a visual control system inside the tank, includes at least a side wall (12), an upper wall (14) and a lower wall (16), the tank (10) comprising a visual inspection system (22) inside the tank (10). The visual inspection system (22) includes an inspection eyepiece (24) arranged in one of the walls (12, 14, 16), a light inlet (26) arranged in one of the walls (12, 14, 16) and configured to allow light from an artificial light source (30) situated outside the tank (10) to enter the tank (10), and a light guide (28) protruding inside the tank (10) from the light inlet (26) to guide the light produced by the artificial light source (30) to the inside of the tank (10).

The present invention relates to an aircraft fuel tank including at least a side wall, an upper wall and a lower wall, the tank comprising a visual inspection system inside the tank.

Such a fuel tank has an environment favorable to the development of microbial pollution. Indeed, over time, water dissolved in the fuel condenses and flows toward the bottom of the tank. This presence of water favors the development of microorganisms such as bacteria, in particular in the calm areas subject to few eddies. When these microorganisms proliferate, they constitute pollution of the tanks. When such proliferation occurs, it becomes necessary to clean the tank, which is expensive and tedious, and immobilizes the aircraft.

To avoid such an inconvenience, it is necessary to detect and prevent the development of microorganisms in the tank, before any proliferation.

### BACKGROUND

To prevent microbial pollution from developing, it is known to withdraw fuel contained in the tank in order to conduct biological contamination tests. These withdrawals are restrictive.

It is also known to perform a visual inspection of the tank. The current (endoscopic) visual inspection methods require completely emptying the fuel tanks in order to be carried out and do not allow a satisfactory inspection. The time necessary for the operation is thus significant, and the inspection visibility is limited by the interior layout of the tank. Lastly, such inspection operations require specialized equipment, in particular equipment qualified for an explosive atmosphere, and must therefore be carried out in a service station equipped with such equipment.

### SUMMARY OF THE INVENTION

The invention aims to improve and facilitate the detection of microbial pollution in an aircraft fuel tank.

To that end, a tank of the aforementioned type is provided, characterized in that the visual inspection system comprises an inspection eyepiece arranged in one of the walls, a light inlet arranged in one of the walls and able to allow light from an artificial light source situated outside the tank to enter the tank, and a light guide protruding inside the tank from the light inlet to guide the light produced by the artificial light source to the inside of the tank.

The tank may further have one or more of the features below, considered alone or according to any technical possible combination:

the light guide comprises a transparent bar extending along a longitudinal axis between a first end connected to the light inlet or forming the light inlet and a free end;

the transparent bar is cylindrical;

the transparent bar is made from polymethyl methacrylate;

the longitudinal axis of the transparent bar is inclined toward the lower wall of the tank;

the transparent bar has, at its free end, a surface transverse to the longitudinal axis of the transparent bar;

the surface transverse to the free end of the transparent bar has a mean profile roughness of less than 0.2 μm;

the transparent bar has a length, considered along the longitudinal axis, greater than 5 cm and advantageously comprised between 10 cm and 20 cm;

the transparent bar has a diameter larger than 10 mm and advantageously comprised between 10 mm and 30 mm;

the eyepiece is arranged in a side wall, the eyepiece being able to allow the observation of at least one region of the lower wall;

the eyepiece is mounted in a first through opening of the wall, tightly relative to the fuel contained in the tank, the light inlet being a porthole arranged in a second through opening of one of the walls of the tank;

the tank includes a zone where the fuel is static, the eyepiece and the light inlet being positioned to allow a visual observation of said zone, the light guide being able to direct the light toward said zone; and

the eyepiece is made from polymethyl methacrylate.

An aircraft comprising a fuel tank as defined above is also provided.

A visual inspection method for an aircraft fuel tank is also provided, including the following steps:

lighting the inside of the tank from an outside artificial light source brought close and pointed toward the light inlet, the light produced by the artificial light source being guided inside the tank through the light guide,

visually inspecting the inside of the tank through the eyepiece, and

treating the inside of the tank as a function of the result of the visual inspection step.

### BRIEF SUMMARY OF THE DRAWINGS

The invention will be better understood upon reading the following description, provided solely as an example, and in reference to the appended drawings, in which:

FIG. 1 is a schematic view of a tank according to an embodiment of the invention;

FIG. 2 is a schematic sectional view of a tank according to an embodiment of the invention; and

FIG. 3 is a flowchart illustrating a visual inspection method for a tank according to an embodiment of the invention.

### DETAILED DESCRIPTION

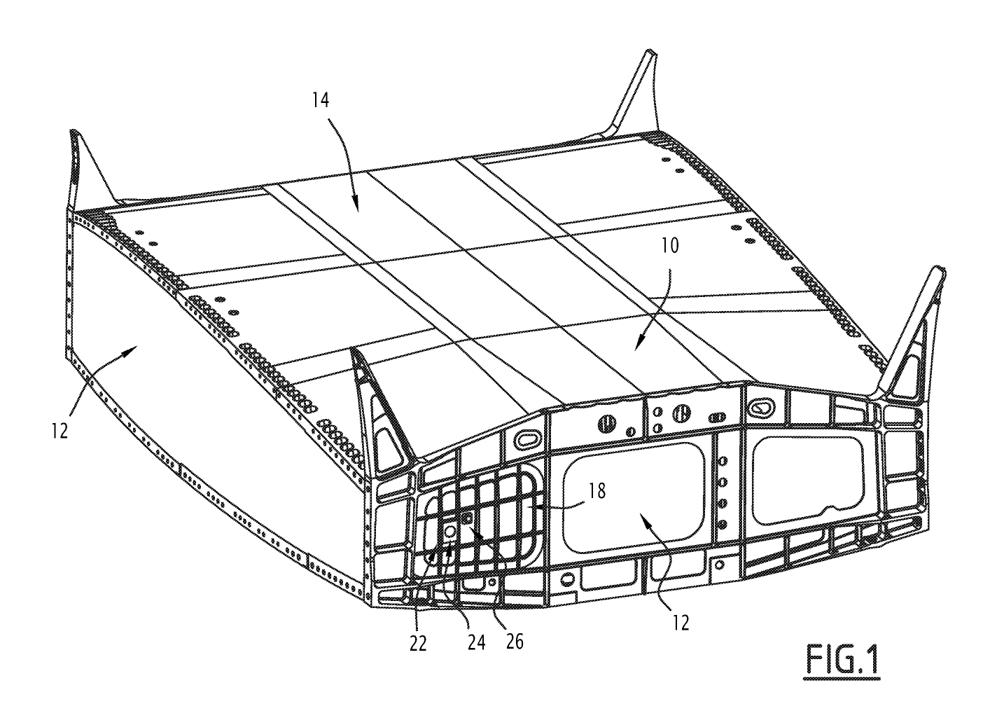

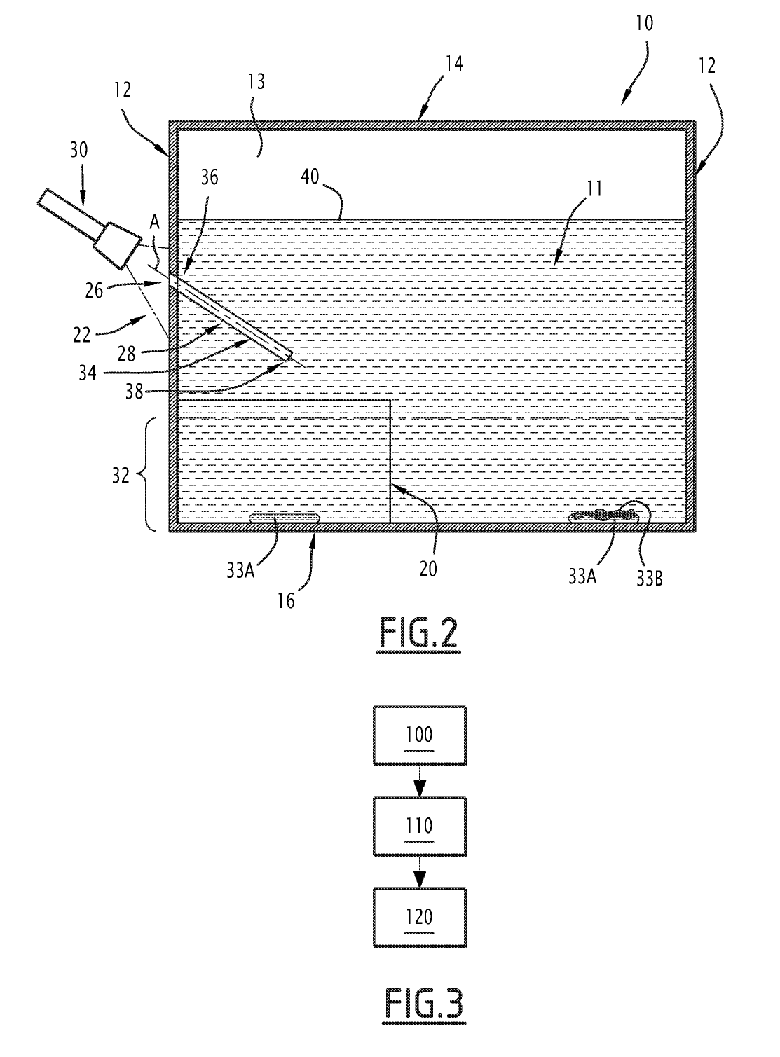

An aircraft tank 10 according to an embodiment the invention is schematically illustrated in FIGS. 1 and 2. The tank 10 defines an inner volume 13 filled with fuel 11.

The tank 10 includes side walls 12, an upper wall 14 and a lower bottom wall 16.

The tank 10 typically comprises an access hatch 18 arranged in one of the walls 12, 14, 16, for example in one of the side walls 12.

The tank 10 includes a collector tank 20, arranged in the inner volume 13 to suction the fuel 11 toward an engine of the aircraft. The collector tank 20 (shown schematically in FIG. 2) protrudes from the lower wall 16 of the tank 10.

The tank 10 also comprises a visual inspection system 22 for the inside of the tank 10.

The visual inspection system 22 includes at least one inspection eyepiece 24, and a light inlet 26 accessible from outside the tank 10. The visual inspection system 22 comprises a light guide 28 protruding in the inner volume 13 from the light inlet 26.

The inspection eyepiece 24 is inserted into one of the walls 12, 14, 16, here a side wall 12, advantageously in the hatch 18. The eyepiece 24 is intended to allow an operator to perform a visual examination of the internal volume 13 of the tank 10 from the outside.

The eyepiece 24 is mounted in a first through opening of the wall 12, tightly relative to the fuel 11 contained in the tank 10.

The eyepiece 24 is for example made from polymethyl methacrylate (in particular marketed under the name plexiglass®).

The light inlet 26 is a porthole arranged in a second through opening of one of the walls 12, 14, 16 of the tank 10, here in the same side wall 12 in which the eyepiece 24 is arranged. The diameter of the inspection eyepiece 24 is preferably greater than 30 mm, in particular comprised between 40 mm and 50 mm, or even more if the structure of the tank 10 allows it, so as to ensure better visibility of the inside of the tank 10, for example by holding an artificial light source 30 in one hand across from the light inlet 28, while keeping one eye across from the inspection eyepiece.

The light inlet 26 is arranged through the hatch 18, next to the inspection eyepiece 24, the light inlet 26 and the inspection eyepiece 24 being close enough to one another for a single person to be able to light the inside of the tank 10 and look at it at the same time.

The light inlet 26 is mounted in the second through opening of the side wall 12, tightly relative to the fuel 11 contained in the tank 10.

The light inlet 26 is able to cause light from an artificial light source 30 situated outside the tank 10 to enter the tank 10. The light source 30 is for example a portable light source transported by the operator performing the visual examination, such as a flashlight, or a portable electronic device, such as a telephone.

Advantageously, the eyepiece 24 and the light inlet 26 are positioned close to the lower wall 16, to allow viewing of the water 33A initially present in the fuel 11 or resulting from the condensation on the walls 12, 14 or 16 and being deposited at the bottom of the tank 10. Indeed, microorganisms 33B, such as bacteria, may develop in this stagnant water.

In the example of the figures, the tank 10 includes a zone 32 where the fuel 11 is static, such a zone 32 for example being situated near the collector tank 20.

The eyepiece 24 and the light inlet 26 are then advantageously positioned to allow a visual observation of the zone 32, across from the latter. The light guide 28 is arranged inside the tank 10.

The light guide 28 is applied outwardly on the light inlet 26. The light guide 28 is able to guide the light from the external light source 30 to the inside of the tank 10.

The light guide 28 comprises a transparent bar 34 extending along a longitudinal axis (A) between a first end 36 connected to the light inlet 26 and a free end 38, arranged protruding in the inner volume 13.

More particularly, the transparent bar 34 is able to guide the light from the light source 30 longitudinally along the longitudinal axis (A).

The bar 34 is for example cylindrical.

The longitudinal axis (A) of the transparent bar 34 is advantageously inclined downward by a non-zero angle relative to the horizontal to be oriented toward the lower wall 16 of the tank 10.

The transparent bar 34 has a length, considered along the longitudinal axis (A), preferably greater than 5 cm and advantageously comprised between 10 cm and 20 cm.

The diameter of the transparent bar 34 is preferably greater than 10 mm, and advantageously comprised between 10 and 30 mm.

The transparent bar 34 is for example made in a single piece from polymethyl methacrylate (in particular marketed under the name plexiglass®).

The transparent bar 34 has a polished peripheral surface.

The free end 38 of the transparent bar 34 is situated below the free surface 40 of the fuel 11, when the tank 10 is full.

The transparent bar 34 has, at its free end 38, a planar surface transverse to the longitudinal axis (A) of the transparent bar 34.

The surface transverse to the free end 28 of the transparent bar 34 is polished. It advantageously has a mean roughness profile (also called roughness “Ra”) below 0.2 μm (measured according to standards NF EN ISO 4287 and NF EN ISO 12085) so as to allow active diffusion of the light inside the tank by this transverse surface.

A visual inspection method for the fuel tank 10 of an aircraft will now be described.

Such a method is for example carried out periodically.

The method includes a lighting step 100, during which an operator provided with an artificial light source 30 brings the source 30 close to the light inlet 26, outside the tank 12, and orients said source.

The light guide 28 then guides the light in the internal volume 13 of the tank 10. The light produced outside the tank 12 by the source 30 therefore enters the internal volume 13 through the transparent bar 34. The light is guided in the bar 34 and primarily exits through the free end 38 of the bar 34.

The lighting of the inside of the tank 10 is thus implemented throughout the entire duration of the inspection.

The method simultaneously includes a step 110 for visually inspecting the inside of the tank 10. During this step 110, the operator performs the visual inspection of the inside of the tank 10 by observing the internal volume 13 of the tank 10 from the outside through the eyepiece 24. He optionally views the presence of water at the lower wall 16 of the tank 10 and/or the presence of colonies of microorganisms 33B.

The interior volume 13 being lit by the light transmitted through the light guide 28, the observation is simple and precise.

Based on the presence or absence of water 33A and/or microorganisms 33B, the method includes a treatment step 120 during which the operator performs a purge operation and/or a biocidal treatment.

The treatment step 120 is for example carried out when the operator observes the presence of microorganisms 33B or when the quantity of water 33A viewed before the observation of microorganisms 33B is above a predetermined quantity.

Alternatively, the transparent bar 34 has a cross-section transverse to the main direction (A) different from the cross-section of a cylindrical cross-section.

Alternatively, the first end 36 of the transparent bar 34 forms the light inlet 26.

Also alternatively, the free end 38 of the transparent bar 34 is situated above the free surface 40 of the fuel 11, when the tank 10 is full.

In another alternative, the visual inspection system 22 includes several inspection eyepieces 24 and/or several light inlets 26 each associated with a light guide 28.

Such a tank 10 thus makes it possible to perform visual inspection operations simply, without specialized tools and by any operator.

The distance separating the inspection eyepiece 24 from the light inlet 26 being small, the same operator places the light source 30 across from the light inlet 26 with one hand and, at the same time, observes the internal volume 13 through the eyepiece 24.

The polishing of the transparent bar 34 makes it possible to guide the light from the light source 30 toward the internal volume 13.

The polishing of the free end 38 of the transparent bar 34 correctly orients the light leaving the transparent bar 34, toward a relevant region of the internal volume 13.

The tank 10 makes it possible to detect and treat the microbial contamination as early as possible, and allows simple viewing of the effectiveness of the biocidal treatments done.

Lastly, such a solution is advantageous inasmuch as it does not require electrical cabling in the internal volume 13, or placement of equipment requiring an electrical power source in contact with the fuel.

Alternatively, the transparent bar 34 is not made from plexiglass, but from any other material able to diffuse the light at its free end and providing mechanical and optical resistance to fuel.

Also alternatively, the transparent bar has a beveled, rounded or conical shape at its free end so as to optimize the diffusion of the light in the tank.

What is claimed is:

1. An aircraft fuel tank including at least a side wall, an upper wall and a lower wall, the tank comprising:
a visual inspection system inside the tank, the visual inspection system comprising:
an inspection eyepiece arranged in one of the side wall, the upper wall or the lower wall,
a light inlet arranged in one of the side wall, the upper wall or the lower wall and configured to allow light produced by an artificial light source situated outside the tank to enter the tank, and
a light guide protruding inside the tank from the light inlet to guide the light produced by the artificial light source to the inside of the tank.

2. The tank according to claim 1, wherein the light guide comprises a transparent bar extending along a longitudinal axis between a first end connected to the light inlet or forming the light inlet and a free end.

3. The tank according to claim 2, wherein the transparent bar is cylindrical.

4. The tank according to claim 2, wherein the transparent bar is made from polymethyl methacrylate.

5. The tank according to claim 2, wherein the longitudinal axis of the transparent bar is inclined toward the lower wall of the tank.

6. The tank according to claim 2, wherein the transparent bar has, at the free end, a surface transverse to the longitudinal axis of the transparent bar.

7. The tank according to claim 6, wherein the surface transverse to the free end of the transparent bar has a mean profile roughness of less than 0.2 μm.

8. The tank according to claim 2, wherein the transparent bar has a length, considered along the longitudinal axis, greater than 5 cm.

9. The tank according to claim 8, wherein the transparent bar has a length, considered along the longitudinal axis, comprised between 10 cm and 20 cm.

10. The tank according to claim 2, wherein the transparent bar has a diameter larger than 10 mm.

11. The tank according to claim 10, wherein the transparent bar has a diameter comprised between 10 mm and 30 mm.

12. The tank according to claim 1, wherein the inspection eyepiece is arranged in the side wall, the eyepiece being configured to allow the observation of at least one region of the lower wall.

13. The tank according to claim 1, wherein the inspection eyepiece is mounted in a first through opening of the side wall, the upper wall or the lower wall, the light inlet being a porthole arranged in a second through opening of one of the side wall, the upper wall or the lower wall.

14. The tank according to claim 1, wherein the inspection eyepiece is made from polymethyl methacrylate.

15. An aircraft comprising the fuel tank according to claim 1.

16. A visual inspection method for a fuel tank, comprising:
providing the fuel tank according to claim 1;
lighting the inside of the tank from the outside artificial light source brought close and pointed toward the light inlet;
visually inspecting the inside of the tank through the inspection eyepiece; and
treating the inside of the tank as a function of the result of the visual inspection.
